(19) United States
(12) Patent Application Publication
(10) Pub. No.: US 2008/0290048 A1
(43) Pub. Date: Nov. 27, 2008

(54) PLASMA SEPARATION DEVICE AND METHOD THEREOF

(75) Inventors: Rainer Jaeggi, Thalwil (CH); Patrick Griss, Otelfingen (CH); Hans-Peter Wahl, Schopfheim (DE)

Correspondence Address: DINSMORE & SHOHL, LLP;ONE DAYTON CENTRE, ONE SOUTH MAIN STREET, SUITE 1300, DAYTON, OH, 45402, US

(73) Assignee: ROCHE DIAGNOSTICS OPERATIONS, INC., Indianapolis, IN (US)

(21) Appl. No.: 12/131,512

(22) Filed: Jun. 2, 2008

(30) Foreign Application Priority Data
Dec. 6, 2005 (EP) 05026571.9
Nov. 1, 2006 (CH) PCT CH2006000612

(51) Int. Cl. B04B 3/00; B01D 33/15; G01N 21/07; G01N 33/48

(52) U.S. Cl. 210/782; 210/198.1; 210/781

(57) ABSTRACT

A device and method thereof for at least partially fractioning or separating fluid from higher density and/or solid particles contained in liquid samples are disclosed. The present invention provides a movable or drivable device having a flow path defined by inner and outer wall surfaces and arranged such that the flow velocity of the liquid sample along the outer wall surface is higher than the flow velocity along the opposite inner wall surface. The flow path provides elements to at least delay the flow of the liquid sample along the outer wall surface. The device is, e.g., suitable for the separation of blood, e.g., of plasma from at least red blood cells, and from red and white blood cells to achieve blood plasma with high purity for analytical reasons.

### CROSS REFERENCE TO RELATED APPLICATIONS

This application is a continuation of International Application No. PCT/CH2006/000612 filed Nov. 1, 2006, which claims priority to EP Application No. 05026571.9, filed Dec. 6, 2005.

### FIELD OF THE INVENTION

The present invention refers generally to fluid separation, and more particularly to a device and a process for at least partially fractioning or separating fluid from higher density and/or solid particles contained in a liquid sample.

### BACKGROUND OF THE INVENTION

For the separation of the serum or plasma from blood as presently disclosed basically a centrifugal test tube, filled with blood is rotated for e.g. twenty minutes at a centrifugal speed of 3000 g. By doing so, one can find all the solid parts of the blood within the sediment and the supernatant liquid consisting out of plasma or serum. Besides this classical blood plasma separation there are known other processes such as e.g. filtration methods. The known filtration methods are not really suitable in microfluidic systems for the separation of plasma out of blood.

For example, Kang et al., Proceedings of the 8^{th} International Conference of Miniaturized Systems in Chemistry and Life (uTAS), Sep. 26-30 (2004), Malmö, Sweden, p. 614, proposes a spiral particle separator in a CD like centrifugal system. Particles are separated by centrifugal force and fluid is pumped by centrifugal acceleration. At the outlet particles are isolated and flow into a waste chamber. Due to priming effects at the first filling of the device, the first fraction of fluid pumped through the device is subject to very low separation efficiency.

Furthermore within U.S. Pat. No. 5,186,844 (Abaxis) micro fluidic structures for the separation of plasma within a rotating disc are disclosed. The layouts are characterized by the separation of particles or cells from the blood in a separation chamber. The plasma is collected within a collecting chamber, which is connected via a fluid outlet port with the separation chamber. The processable volume of blood is defined by the dimension of the sedimentation chamber and the position of the fluid outlet port, which means that the volume to be processed is very limited.

Brenner et al., Proceedings of the 8^{th} International Conference of Miniaturized Systems in Chemistry and Life (uTAS), Sep. 26-30 (2004), Malmö, Sweden, p. 566, again proposes fluidic structures which are very similar to the design layouts as disclosed within the above mentioned U.S. Pat. No. 5,186,844. The separation of parts within the blood is executed within a micro fluidic canal section (drain channel) and a decant chamber. Again the range of volume to be processed is very limited.

Blattert et at., Microfluidics, BioMEMS, and Medical Microsystems II, Proceedings of SPIE, Vol. 5345, 17 (2003), proposes a method and a device for the separation of plasma by using centrifugal force within an arcuated non rotating canal. The achieved separation efficiency can be compared with the so called “plasma skimming” process without using any centrifugal force. The purity of the plasma achieved by using this method is very limited.

C. Bor Fuh, Analytical Chemistry, Apr. 1, 2000, pp. 266A-271A, proposes a splitting technique for the separation of particles and cells by utilizing the different physical properties of particles or cells under the influence of centrifugal forces.

The U.S. Pat. Appln. Pub. 2002/0068675 A1 a centrifugal separation device for use in a fluid separation system is disclosed. A composite fluid to be separated is delivered to a fluid receiving area, from which it travels to a circumferential fluid separation channel, which separates the composition into components which each then travel to distinct fluid outlet channels. The individual fluid components are then moved to separate collecting bags.

In the U.S. Pat. No. 6,635,163 a separation device is disclosed, where the separation of a multi-component substance containing molecules of different sizes is achieved by narrowing or enlarging the diameter of a flow-pass, through which the molecule mixture is transported. The separation is achieved due to the molecule size dependence of the entropic trapping effect.

All the above disclosed rotating micro fluidic systems or separation methods respectively cannot be operated with any or arbitrary volume of blood. This can be either due to the dimensions of the device or the structures respectively or due to problems at the priming procedure which means at the first filling of the devices. In other words, the blood volumes are very limited.

Furthermore the above described prior art methods and structures are either not feasible in a continuous flow or require a minimum volume of blood or the result is a non-complete separation or an insufficient separation of blood cells and the plasma.

### SUMMARY OF THE INVENTION

It is against the above background that the present invention provides a more reliable and easier processable device for the separation of plasma or serum from blood, or more generally, for the fractioning or separation of a fluid from higher density and/or solid particles contained in a liquid sample.

In one embodiment, the present invention discloses a more sophisticated and more reliable methods for the separation of plasma from blood for the use in microfluidic systems to enable e.g. continuous or further processing of the separated samples.

In another embodiment, the present invention also integrates the separation step into an analytical device for which using known filtration methods is not possible.

In one embodiment, disclosed is a device or an arrangement for at least partially separating fluid from higher mass-density and/or solid particles contained in a liquid sample such as, e.g., blood plasma to be separated at least from red blood cells to get a red blood cell free fluid fraction for, e.g., analysis purpose. For that purpose a device or an arrangement is disclosed which can be driven or moved such, that a fluid flowing within a fluid path in or on the device is forced to flow by pressure force such as, e.g., centrifugal force, gravity force etc., the fluid path being arranged such that at least one force component is not parallel to the direction of the flow path of the fluid. The device comprises e.g. a rotatable plate like or disc-like body in or on which at least one flow path is integrated or arranged in which at one internal wall surface the flow velocity of the sample fluid is higher compared with the velocity at e.g. the opposite internal wall surface to enable separation or sedimentation out of the fluid sample of solid particles or particles with higher mass-density than the density of the liquid. In one embodiment, the distance between the flow path and the rotation axis of the rotatable plate or disc-like body is at least partially increasing or constant. The path can be e.g. an arcuated ring like, helical or spiral separation or sedimentation path or channel for the fluid, which is arranged in or on the body, the path or channel comprises at least along a section of the mentioned one wall surface against which the nonparallel force component is directed, resistive elements for the reason that at least the flow of the sample fluid mixture is delayed along the mentioned one wall surface section. With other words at the mentioned wall surface as e.g. the outer wall surface, means are arranged or incorporated, which influence the flow velocity of the fluid sample and/or which are enabled to capture parts of the fluid sample, such as solid parts and/or particles with a higher mass-density than the liquid.

The mentioned one wall surface of the path or channel, which in fact is a separation or sedimentation path or channel is designed such, that the flow rate is delayed along the mentioned wall surface and a separation or sedimentation of the high density and/or solid particles from the remaining fluid occurs along the wall surface.

According one embodiment the wall comprises at least along parts of the outer wall surface successively arranged cavities for the collection of the higher density and/or solid particles such as e.g. the red blood cells and additional solid parts of the blood.

Other embodiments of the mentioned wall surface are possible such as e.g. the definition of wave forms in the outer wall surface, the formation of a zigzag behaved surface, the arrangement of capture cavities, pocket volumes, etc.

According to one embodiment the path or channel can be arranged within the disc-like body in a spiral or helical form such, that towards the rotor axis of the device a fluid mixture input zone is arranged and that the path or channel from the input zone is defining a helical path towards the outer periphery boundary of the disc-like body. In direction to the periphery of the disc-like body of the device discharge conducts can be arranged near the inner and/or the outer wall of the path or channel respectively to discharge either the fluid such as e.g. the blood plasma or the higher density and/or solid particles, such as for instance the red and white blood cell particles.

According to one embodiment the helical like path or channel comprises successively arranged cavities as resistive elements along the outer wall surface, the total volume of the cavities or elements respectively is such, that at least an essential part or preferably almost all of the higher density and/or solid particles can be collected, such that at least almost all of the fluid such as the plasma volume can be used for further analysis purpose.

The resistive elements along the outer path or canal wall are such, that the higher density and/or solid particles are collected within the resistive elements and that an overflow of the collected higher density and/or solid particles may be prevented. Specific and preferred designs of the cavities or restrictive elements shall be described in more details with reference to the attached figures; the description will follow later on within this description.

According to a further embodiment, the helical path or channel respectively comprises channels, ducts, bypasses, and the likes to remove plasma or to remove higher density and/or solid particles such as for instance red and white blood cells.

Again according a further embodiment the diameter of the path or channel is decreasing along the path length, to take on one side the separated volume of the high viscous and solid particles into consideration and further more by decreasing the cross section of the channel along the pass. As a consequence, the flow resistance will increase so that at equal centrifugal acceleration the flow of the liquid sample or blood respectively shall decrease, and therefore the efficiency of sedimentation or separation of higher density and solid particles will increase.

As already described above during the radial and/or helical flow towards the outside periphery of the device of the present invention, a separation of the fluid mixture occurs resulting in a more or less solid free fluid such as, for instance, a cell free blood plasma for analysis purpose.

The invention shall be described in more details with reference to the examples, shown within the attached figures.

### BRIEF DESCRIPTION OF THE DRAWINGS

FIG. 1 shows in perspective view an inventive disc-like device comprising a helically arranged separation path with an outer wall surface comprising resistive structures,

FIG. 2 shows in perspective view an inventive device equivalent to a disc segment out of a disc as shown in FIG. 1,

FIG. 3 shows in perspective view a further design of a plate like device, comprising an arcuated separation path,

FIG. 4 shows in a sectional view one specific design of the resistive elements of the outer wall surface of a separation channel,

FIGS. 5a-5f show the section A out of FIG. 1, showing a sectional part of the separation channel with different designs of the outer wall comprising the resistive structures,

FIG. 6 shows a further embodiment of the separation channel from FIG. 1,

FIG. 7 shows in perspective view again a further embodiment of an analytical separation device, comprising a separation path with a plurality of collecting channels,

FIG. 8 shows in perspective view a further embodiment of a plate like device, comprising a separation channel with a plurality of collecting channels,

FIG. 9 again shows in perspective view a further embodiment of a disc-like device, comprising a reticulated separating or sedimentation path,

FIG. 10 shows again a further embodiment of a rotatable device comprising a separation or sedimentation path with a plurality of collecting paths, and

FIG. 11 shows a further embodiment of a device layout, comprising a device geometry, enabling the device being integrated into an arrangement with further elements or devices.

### DETAILED DESCRIPTION

FIG. 1 shows in perspective view an inventive disc-like device 1 rotatable around a central rotation axis ω. The disc-like device 1 can have the size of a conventional compact disc or can be of smaller or larger size. Within the disc a separation path or channel 3 is arranged which is extending from a central area of the device helically towards the periphery border of the disc-like device. The helically arrangement is represented by the radius or distance R₁ of the path near to a feeding zone and a second larger radius or distance R₂ in direction to the border of the device. To describe the invention in more details the section A is shown in enlargement and in greater details in the following FIGS. 5a to 5f.

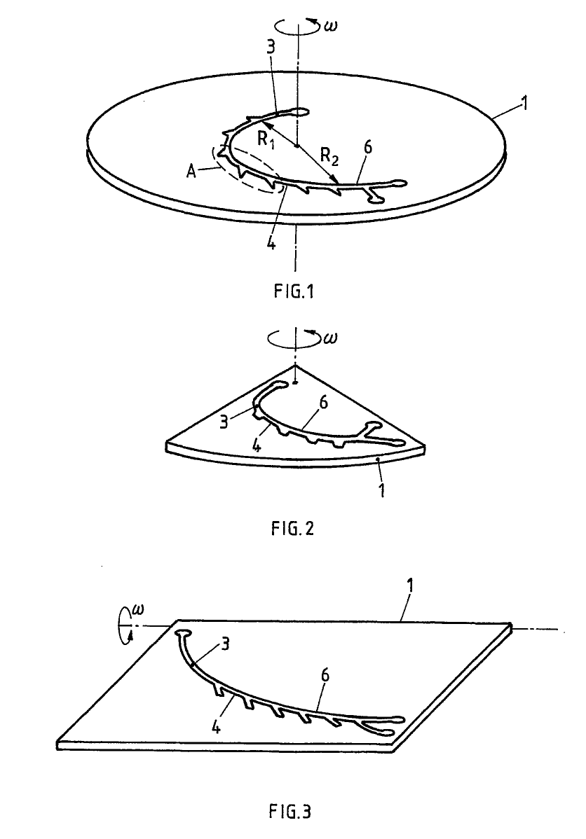

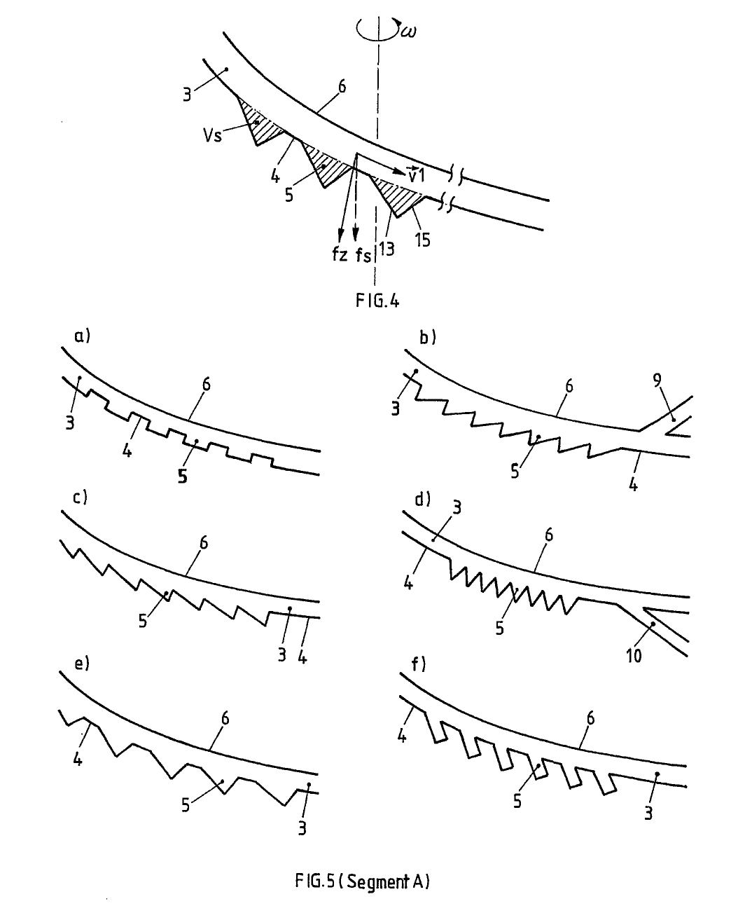

FIG. 2 shows a further embodiment of an inventive device 1, comprising a segment of a disc in rotatable around a displaced arranged rotation axis ω. Again on this disc segment a helically designed separation or sedimentation channel 3 is arranged, the design of the channel being described in more details with reference to the following FIGS. 4 and 5a to 5f.

In FIG. 3 schematically a plate like analytical device 1 is shown, being rotatable around a rotation axis ω, being arranged along a side edge of the plate like device 1. Of course the rotation axis can also be at another location arranged on the plate like device 1. On the plate 1 equivalent to FIGS. 1 and 2, a separation or sedimentation path 3 is arranged, comprising an outer wall surface 4 and an inner wall surface 6, seen in direction of the rotation of the plate like device 1.

In FIG. 4 a segment of the sedimentation or separation path 3 is shown in enlargement to explain the basic idea of the present invention. The sedimentation or separation path 3, as known out of the devices according to FIGS. 1 to 3, does have an outer surface 4 and an inner surface 6, along which, the fluid sample to be separated is flowing with the velocity vl. The fluid is forced in the flow direction with the velocity vl due to the rotation of the device. Rotation is one possibility to force the sample to flow, but any other force, such as e.g. gravity, can be used to force the sample flowing through the channel 3. Along the surface wall 4, due to the centrifugal force fz, the liquid sample does have a higher flow velocity, than along the opposite inner wall surface 6. Due to the flow and the rotation the resulting force is the so called Coriolis force fs, which together with the centrifugal force urges solid particles or particles with a higher mass-density than the liquid, to sediment out of the liquid, in direction to the outer wall surface 4. To capture these high density particles or solid particles along the outer wall surface 4 it is disclosed, according to the present invention, to arrange means or elements 5 to capture the high density or solid particles. To optimize the capture or collection of the high density or solid particles, these elements 5, such as e.g. triangular resistive structures are such, that the retention of the particles, such as e.g. cells within the element 5 is optimal or maximal respectively. On the other hand the amount of elements should be such, that an overfilling or overflow of centrifuged particles can be prevented.

Important and responsible for the optimization of the restrictive elements or structures 5 is the volume Vs as well as the angle of the retaining wall 15 of the resistive elements 5. The volume Vs of one element is characterized by the mentioned angle θ and the lengths or heights of the two legs 13 and 15 of the resistive element. Furthermore of importance of course is also the geometry of the channel which means the width and the depth of the channel as well as the radial position of the channel and the angle between the channel axis and the radius which means the distance to the rotation axis of the disc-like device.

FIG. 5a to 5f show embodiments of the section A out of the separation channel 3 arranged within the disc-like device 1 of FIG. 1. The separation path or channel 3 according FIG. 5a shows an inner surface wall 6 which is at least almost even and/or bent, while the outer surface 4 is uneven which means does include resistive elements 5. The resistive elements or structures 5 are arranged on the outer wall 4 of the separation channel 3 which means on the wall, which is arranged in direction to the centrifugal force. The structures do have the function of resistive elements which should ensure, that high density or solid parts, which means in the case of blood the cellular contents are held back within the resistive elements. As a result occurs the separation of the fluid from the high viscous or solid particles which means in the case of blood of the blood plasma from the blood cells.

In FIG. 5b, a further embodiment of the resistive elements 5 is shown, which may be appropriate or suitable for holding back the solid particles out of the sample mixture. By arranging bypasses or branching off channels such as e.g. the bypass 9 as shown in FIG. 5b the fluid such as e.g. the plasma can be separated from the sample mixture.

FIGS. 5c, 5d, 5e and 5f show further embodiment of the resistive elements 5.

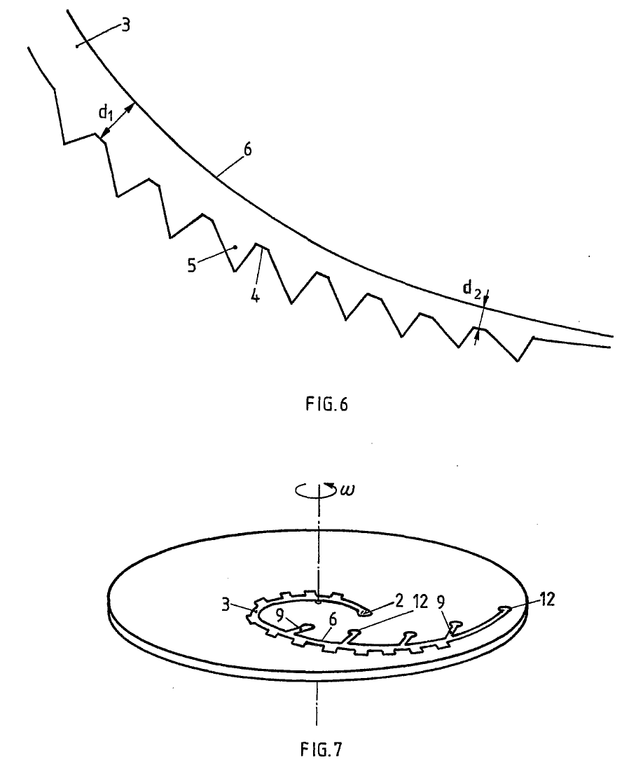

FIG. 6 shows a further embodiment of a separation channel 3 comprising an inner channel wall 6 which is almost even and with an outer surface 4 including resistive elements 5. The design according to FIG. 6 is such that along the path the cross section of the channel is decreasing which means the diameter d, is bigger than the diameter d₂ seen in successional direction of the channel. The total volume Vs which is defined by the volume of the individual structure volumes corresponds in the ideal case to the centrifuged and retained total amount of solid particles which means in the case of blood to the total volume of the sedimented cells. The decreasing of the canal cross section in flow direction results in an increase of the flow resistance. As a result the flow of the sample mixtures which means of the blood will decrease at an equal centrifugal acceleration which means without decreasing the rotation frequency, so that the sedimentation and separation efficiency shall be increased. The reason for this effect is due to the slowing down flow so that more time is available for sedimentation and centrifugation.

In case, that the total volume Vs of the resistive elements is sufficient, as e.g. in case of blood a plasma can be achieved at the end of the channel containing practically no cells anymore within the plasma, without the need of any bypasses or branching off channels. Practically any small volume of blood can be introduced within the channel for gaining cell free plasma. At bigger blood volumes the canal section including resistive elements should be elongated and eventually bypasses or branching off channels should be used to remove the plasma out of the sample mixture.

In FIGS. 7 to 10 further designs for separation channels are shown the use of bypasses or branching off channels for the separation of plasma, so that e.g. reduced blood and cell containing blood can be collected. For the reason of simplification only resistive elements are shown in the outer most arranged separation channels.

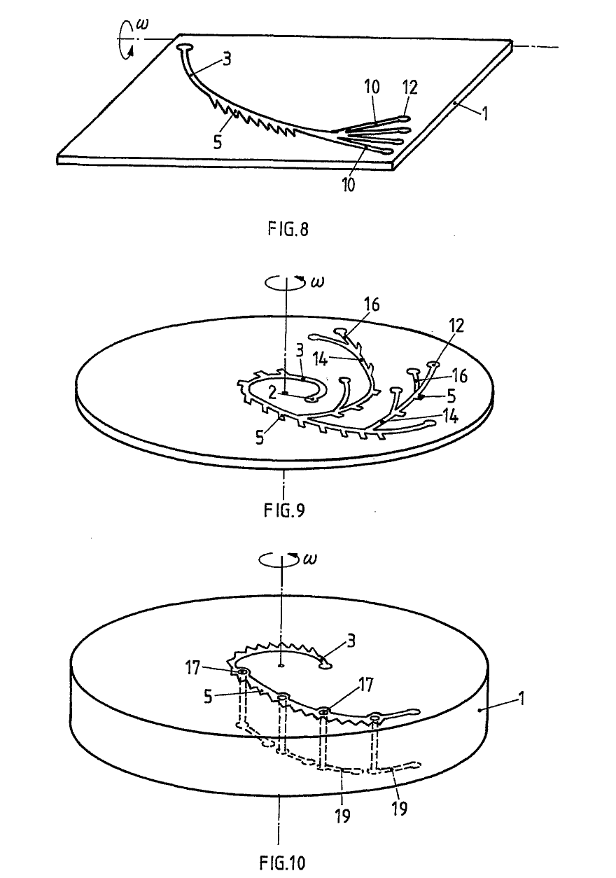

FIG. 7 shows the sequential arrangement of bypasses or branching off channels 9, while FIG. 8 shows the arrangement of parallel branching off channels 10 at the end of the separation path 3. In collecting zones 12 the separated samples or liquids can be collected or removed respectively.

FIG. 9 again shows a cascade arrangement of bypasses or branching off channels 14 and 16 for gaining plasma with increasing purity of the plasma.

FIG. 10 finally shows branching off channels 19, which are arranged through holes 17 out of the plane within the disc-like device 1 in which the separation path 3 is arranged.

One advantage of the designs as shown in FIGS. 1 to 8 is that any amount or volume of a sample such as a liquid as in particular of blood can be processed and any ratio of separation out of any possible small amounts of the sample such as out of blood can be achieved. In addition, problems which may occur in existing separation chambers such as e.g. mentioned in the U.S. Pat. No. 5,186,844 occurring at the interface layer between liquid and solid particle section, e.g. due to the existence of blood platelets or blood cells can be avoided due to the relatively small dimensions of the resistive elements. Furthermore an additional advantage is that the cell/particle separation can be done continuously which means no special collection or separation chambers must be used.

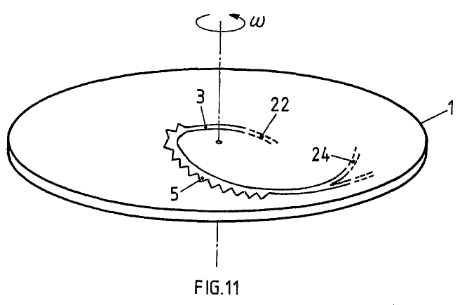

In FIG. 11 finally it should be shown schematically that the inventive device can also be used as one element within a larger arrangement for the separation or sedimentation parts out of a liquid sample. Schematically indicated, the interface to a preceding device such as the introduction of the liquid sample is shown by dashed lines 22 near the rotation axis ω of the device 1, while again by dashed lines in sections 24 the separated or purified liquid or liquid sample respectively, can be introduced into a further following device. The preceding device can be e.g. a fluid metering device or a mixing device for a plurality of fluids. The following device could be e.g. a mass spectro-metric device, a device for electrophoresis analysis, for photometric measurements, for fluorescence measurements, bio/chemical luminescence, electrochemical detection, etc. But again for the sedimentation or separation of parts of the liquid sample collecting or resistive elements 5 are arranged along that wall surface of the sedimentation path 3, along which the velocity of the sample is higher than along the opposite wall surface of the path.

The embodiments shown in FIGS. 1 to 11 only represent possible examples which can be changed and modified in any different way. Of mayor importance is, that on a removable or drivable device or body such as a rotatable device, such as e.g. the disc or plate like device as shown in FIGS. 1 to 8, a separation channel or path is arranged which comprises at its outer wall surface captive or resistive elements to reduce the flow speed along the outer surface wall to increase the separation or sedimentation of any solid or high density particles in the sample mixture. In the case of blood the separation of blood plasma from cell particles such as red and white blood cells can be achieved so that blood plasma can be used e.g. for further analysis steps.

What is claimed is:

1. A device for at least partially separating fluid from higher density and/or solid particles contained in a liquid sample, the device comprising a moveable body about an axis of rotation, the body providing at least one flow path for the liquid sample, the flow path having an outer wall surface and an opposed inner wall surface, the inner wall surface being located closer to the axis of rotation than the outer wall surface, wherein the outer and inner wall surfaces are configured such that, when moving the body about the axis of rotation, flow velocity of the liquid sample along the outer wall surface is higher than the flow velocity along the inner wall surface, and the flow path further provides elements to at least delay the flow of the liquid sample at least along the outer wall surface.

2. The device according to claim 1 wherein the moveable body is a disc-like body.

3. The device according to claim 1 wherein the moveable body is a plate-like body.

4. The device according to claim 1, wherein the flow path is an arcuated circle.

5. The device according to claim 1, wherein the flow path is a spiral.

6. The device according to claim 1, wherein the flow path is a helically arranged separation path.

7. The device according to claim 1, wherein the flow path has a first portion with a first radius from the axis of rotation and a second portion with a second radius from the axis of rotation which is larger than the first radius.

8. The device according to claim 1, wherein the flow path has a first width that narrows to a second width.

9. The device according to claim 1, wherein the elements are provided successively along the outer wall surface.

10. The device according to claim 1 wherein the elements each provide a wall surface against which at least one flow force component is directed at when moving the body.

11. The device according to claim 1 wherein the elements are resistive elements.

12. The device according to claim 1 wherein the elements are successively arranged cavities.

13. The device according to claim 1 wherein the elements are cavities having a shape selected from square like, triangle like, and rounded recesses.

14. The device according to claim 1, wherein the elements are successive cavities arranged in a wave like form.

15. The device according to claim 1, wherein the flow path further provides an input zone for the liquid sample adjacent the axis of rotation and at least one collection zone adjacent a periphery of the body.

16. The device according to claim 1 wherein the flow path further provides at least one bypass channel.

17. The device according to claim 1 wherein the flow path further provides at least one bypass channel providing another flow path with additional ones of the elements.

18. The device according to claim 1 wherein the flow path further provides at least one bypass channel providing another flow path with additional ones of the elements and arranged along the inner wall surface.

19. The device according to claim 1 wherein the flow path provides a plurality of bypass channels arranged along the inner wall surface and each providing additional ones of the elements.

20. The device according to claim 1 wherein the flow path provides at least one hole between the outer and inner will surfaces, wherein the hole connects to a bypass channel provided out of plane from the flow path.

21. The device according to claim 1 wherein the flow path provides a plurality of bypass channels each arranged to collect or discharge fluid with different levels of at least one of purity, density, and solid particles.

22. The device according to claim 1 wherein flow path is configured to interface with a second body of another device.

23. The device according to claim 1 wherein the flow path is provided in the body.

24. The device according to claim 1 wherein the flow path is provided on the body.

25. A method for at least partially separating fluid from higher density and/or solid particles contained in a liquid sample, the method comprising:
providing the liquid sample to a device comprising a body moveable about an axis of rotation, the body providing at least one flow path for the liquid sample, the flow path having an outer wall surface and an opposed inner wall surface, the inner wall surface being located closer to the axis of rotation than the outer wall surface, and the flow path further provides elements to at least delay the flow of the liquid sample at least along the outer wall surface; and
moving the body about the axis of rotation such that the liquid sample is forced to flow through the at least one flow path and that at least part of the higher density and/or solid particles are collected along at least sections of outer wall surface of the flow path, in which the flow velocity is higher than along the opposite inner wall surface of the flow path, to provide the fluid as an at least partially purified liquid.

26. The method according to claim 25 wherein moving the body forces the liquid sample through the flow path which has a shape selected from an arcuated path, a helical path, a spiral like path, a path with at least partially increasing distance to the axis of rotation, and a path with at least a section with a constant distance to the axis of rotation.

27. The method according to claim 25 wherein the body is a plate like or disc-like rotatable body.

28. The method according to claim 25 wherein the elements are arranged to collect, capture, or sediment portions of the liquid sample.

29. The method according to claim 25 wherein at least part of the collected high density particles and/or solid particles and/or at least part of the at least partially purified liquid are collected within bypasses or branching off channels which are either in connection with the elements or the inner wall surface.

30. The method according to claim 25 wherein the liquid sample is blood which is separated along the flow path into an at least almost blood free plasma and blood.

31. The method according to claim 30 wherein the blood plasma is further separated along the flow path to be mostly free of any blood cell particles.

32. Use of the device according to claim 1 for the separation of blood.
